(19) United States
(12) Patent Application Publication
(10) Pub. No.: US 2026/0232961 A1
(43) Pub. Date: Aug. 13, 2026

(54) CATHETER HANDLE FRICTION BRAKE

(71) Applicant: Boston Scientific Scimed, Inc, Maple, MN (US)

(72) Inventors: James Holmberg, Champlin, MN (US); Allison Michelle Marsh, Minneapolis, MN (US); Michael Strawn, Minneapolis, MN (US)

(21) Appl. No.: 19/535,641

(22) Filed: Feb. 10, 2026

Related U.S. Application Data
(60) Provisional application No. 63/756,568, filed on Feb. 10, 2025.

(51) Int. Cl. A61M 25/01; A61B 18/14; A61M 25/00

(52) U.S. Cl. CPC A61M 25/0136; A61B 18/1492; A61M 2025/0006

(57) ABSTRACT

A friction brake for use in a medical device includes a polymeric body having a first arm and a second arm separated by a slot. The slot has a first open end and a second closed end. The first arm and the second arm are connected by a hinge adjacent the second end of the slot. A channel configured to receive an elongated medical device traverses the slot. The polymeric body is configured to apply constant friction force to the elongated medical device and is formed of a low friction material configured to minimize any stick-slip phenomenon between the polymeric body and the elongated medical device.

### CROSS REFERENCE TO RELATED APPLICATIONS

This application claims priority to U.S. Provisional Patent Application No. 63/756,568 entitled, “CATHETER HANDLE FRICTION BRAKE,” filed Feb. 10, 2025, which is hereby incorporated by reference in its entirety.

### TECHNICAL FIELD

The present disclosure relates to medical systems and methods for controlling the delivery and positioning of medical devices during medical procedures. More specifically, the present disclosure relates to medical systems and methods for controlling the deployment, shape, and/or arrangement of medical devices during medical procedures.

### BACKGROUND

Various medical procedures involve elongated medical devices, such as catheters, sheaths, guidewires, stylets, and dilators, inserted into a patient's vasculature. In certain procedures, the elongated medical device may be navigated through the vasculature to a target location in the body. The distal end of the catheters may be inserted into the patient's heart chambers in, for example, interventional electrophysiology procedures. The distal end of the catheter may include one or more electrodes that are used to delivery therapy (e.g., ablation) or map the surface of the heart tissue (e.g., identify the locations of heart tissue that are a source of the arrhythmias). Accurately steering, locating, and positioning a location of the elongated medical device, including the distal end portion of the elongated medical device, is necessary. In some systems, a handle includes an actuator for changing the shape or arrangement of the distal end portion of the elongated medical device. However, these actuators can be cumbersome, requiring dexterity to disengage the actuator by pressing down on the actuator prior to moving.

### SUMMARY

Example 1 is a friction brake for use in a medical device. The friction brake includes a polymeric body having a first arm and a second arm separated by a slot. The slot has a first open end and a second closed end. The first arm and the second arm are connected by a hinge adjacent the second end of the slot. A channel traversing the slot is configured to receive an elongated medical device. The polymeric body is configured to apply constant friction force to the elongated medical device.
Example 2 is the friction brake of Example 1, further comprising a clamp configured to hold the polymeric body.
Example 3 is the friction brake of Examples 1 or 2, wherein the channel comprises a circular cross-section.
Example 4 is the friction brake of any of Examples 1 to 3, wherein the clamp includes a portion having a circular cross-section.
Example 5 is the friction brake of any of Examples 1 to 4, wherein the polymeric body includes a cylindrical portion.
Example 6 is the friction brake of any of Examples 1 to 5, wherein the polymeric body is formed of a low friction material configured to minimize any stick-slip phenomenon between the polymeric body and the elongated medical device.
Example 7 is the friction brake of any of Examples 1 to 6, wherein the material comprises acetal.
Example 8 is the friction brake of any of Examples 1 to 7, wherein the hinge protrudes above a surface of the polymeric body.
Example 9 is the friction brake of any of Examples 1 to 8, further comprising a flare adjacent the first open end of the slot.
Example 10 is the friction brake of any of Examples 1 to 9, wherein the first arm and the second arm include an outer surface having a straight portion and a curved portion.
Example 11 is the friction brake of any of Examples 1 to 10, wherein the polymeric body further comprises a protrusion positioned adjacent the channel and extending into the slot.
Example 12 is the friction brake of any of Examples 1 to 11, wherein the clamp includes at least one wing that extends away from the polymeric body.
Example 13 is the friction brake of any of Examples 1 to 7, wherein the polymeric body includes a groove configured to receive the clamp.
Example 14 is the friction brake of any of Examples 1 to 7, or 13, wherein the polymeric body includes an outer dimeter that tapers along a longitudinal axis of the channel.
Example 15 is the friction brake of any of Examples 1 to 7, 13, or 14, wherein the channel passes through the hinge.
Example 16 is a friction brake for use in a medical device. The friction brake includes a polymeric body having a first arm and a second arm separated by a slot. The slot has a first open end and a second closed end. The first arm and the second arm are connected by a hinge adjacent the second end of the slot. A channel configured to receive an elongated medical device traverses the slot. The polymeric body is configured to apply constant friction force to the elongated medical device and is formed of a low friction material configured to minimize any stick-slip phenomenon between the polymeric body and the elongated medical device.
Example 17 is the friction brake of Example 16, further comprising a clamp configured to hold the polymeric body.
Example 18 is the friction brake of Example 16, wherein the channel comprises a circular cross-section.
Example 19 is the friction brake of Example 16, wherein the clamp includes a portion having a circular cross-section.
Example 20 is the friction brake of Example 16, wherein the polymeric body includes a cylindrical portion.
Example 21 is the friction brake of Example 16, wherein the material comprises acetal.
Example 22 is the friction brake of Example 16, wherein the hinge protrudes above a surface of the polymeric body.
Example 23 is the friction brake of Example 16, further comprising a flare adjacent the first open end of the slot.
Example 24 is the friction brake of Example 16, wherein the first arm and the second arm include an outer surface having a straight portion and a curved portion.
Example 25 is the friction brake of Example 16, wherein the polymeric body further comprises a protrusion positioned adjacent the channel and extending into the slot.
Example 26 is the friction brake of Example 17, wherein the clamp includes at least one wing that extends away from the polymeric body.
Example 27 is the friction brake of Example 17, wherein the polymeric body includes a groove configured to receive the clamp.
Example 28 is the friction brake of Example 16, wherein the polymeric body includes an outer dimeter that tapers along a longitudinal axis of the channel.
Example 29 is the friction brake of Example 16, wherein the channel passes through the hinge.
Example 30 is an ablation device. The ablation device includes a handle including an actuator. An outer member includes a proximal portion connected to the handle and a distal portion having at least one electrode. An inner member is movable within the outer member using the actuator. A shape of the distal portion is changeable by moving the inner member. A friction brake includes a polymeric body. The polymeric body has a first arm and a second arm separated by a slot. The slot has a first open end and a second closed end. The first arm and the second arm are connected by a hinge adjacent the second end of the slot. A channel traverses the slot and the inner member is received in and movable through the channel. The polymeric body is configured to apply constant friction force to the inner member such that a position of the actuator is maintained.
Example 31 is the ablation device of Example 30, wherein the friction brake includes a clamp configured to hold the polymeric body.
Example 32 is the ablation device of Example 30, wherein the distal portion is changeable from a stored configuration to an expanded configuration.
Example 33 is the ablation device of Example 30, wherein the distal portion is changeable from a straight configuration to a curved configuration.
Example 34 is a handle for use in a medical procedure. The handle includes a housing having an outer surface and an inner cavity. An actuator is moveable relative to the housing and is configured to translate an elongated medical device. A friction brake located in the inner cavity. The friction brake includes a polymeric body. The polymeric body has a first arm and a second arm separated by a slot. The slot includes a first open end and a second closed end. The first arm and the second arm are connected by a hinge adjacent the second end of the slot. A channel traverses the slot that receives the elongated medical device. The polymeric body is configured to apply constant friction force to the elongated medical device such that a position of the actuator is maintained.
Example 35 is the handle of Example 34, comprising at least one column configured to support the friction brake within the inner cavity.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a perspective view of a handle for use with a medical device, in accordance with embodiments of the disclosure.

FIG. 2A is a perspective view of a distal portion of an ablation catheter in a stored configuration, in accordance with embodiments of the disclosure.

FIGS. 2B and 2C illustrate perspective views of a distal portion of an ablation catheter in various expanded configurations, in accordance with embodiments of the disclosure.

FIG. 3 is a side view of the handle in FIG. 1 with a portion of the housing missing, in accordance with embodiments of the disclosure.

FIG. 4 is a perspective view of the handle in FIG. 1 with a portion of the housing missing, in accordance with embodiments of the disclosure.

FIG. 5 is a perspective view of first embodiment of a friction brake for use with the handle of FIG. 1.

FIGS. 6A and 6B are exploded perspective views of the friction brake of FIG. 5.

FIG. 7 is a perspective view of second embodiment of a friction brake for use with the handle of FIG. 1.

FIG. 8 is a perspective view of third embodiment of a friction brake for use with the handle of FIG. 1.

FIG. 9 is a perspective view of fourth embodiment of a friction brake for use with the handle of FIG. 1.

FIG. 10 is a perspective view of fifth embodiment of a friction brake for use with the handle of FIG. 1.

FIG. 11A-11D are perspective views of various clamps for use with a friction brake, in accordance with embodiments of the disclosure.

FIG. 12 is a perspective view of an embodiment of a friction brake for use with the handle of FIG. 1 utilizing a collet.

FIGS. 13A and 13B are cross-sectional views of the components of the friction brake of FIG. 12, in accordance with embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

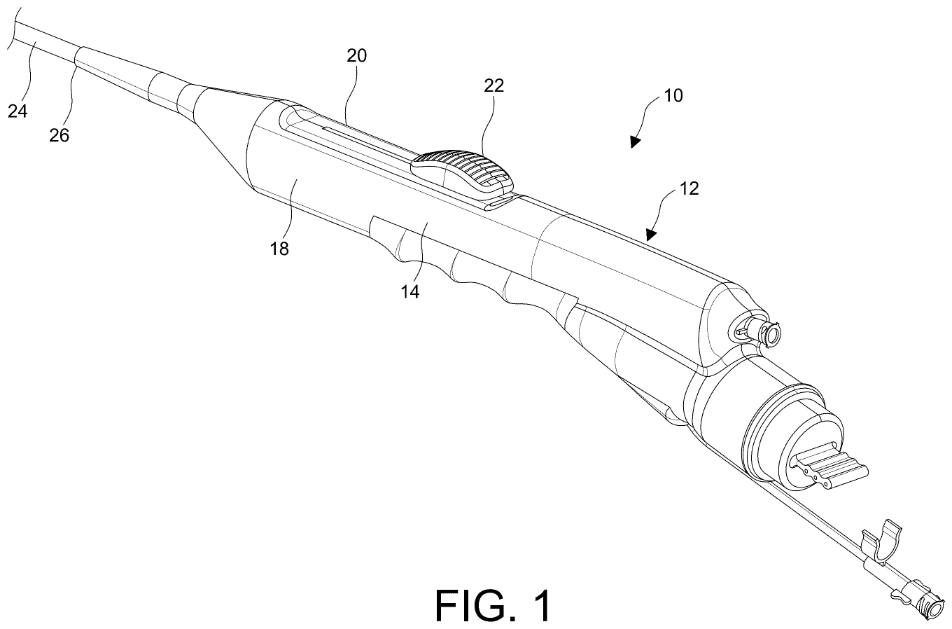

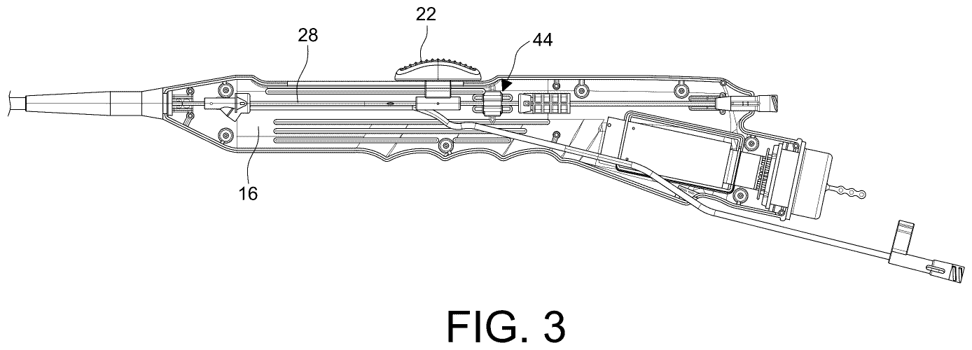

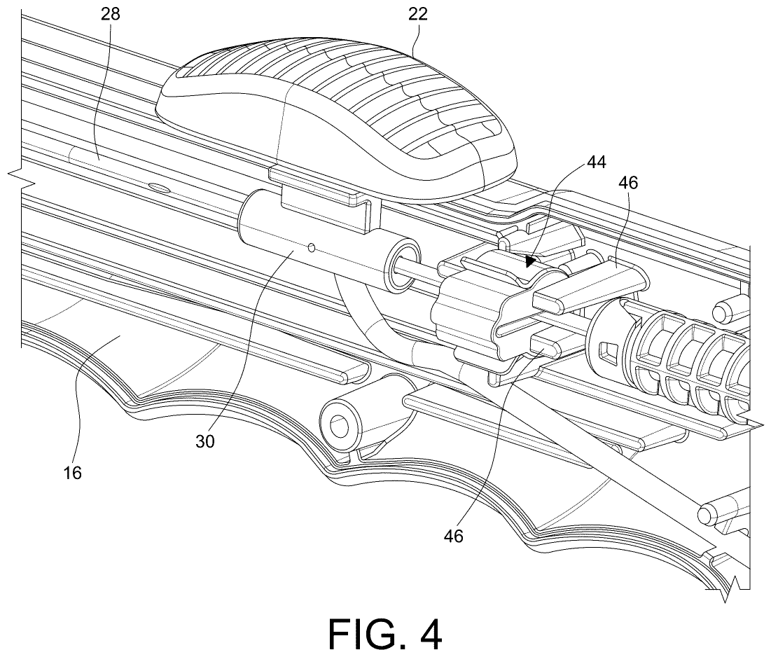

FIG. 1 is a perspective view of a handle 10 for use with a medical device, in accordance with embodiments of the disclosure. The handle 10 includes a housing 12 having an outer surface 14 and an inner cavity 16 (illustrated in FIGS. 3 and 4). The housing is formed of a left housing portion 18 and a right housing portion 20 joined together to create the inner cavity 16. An actuator 22 is moveable relative to the housing 12 and is configured to translate a portion of an elongated medical device. The elongated medical device is configured to change shape or arrangement by manipulation of the actuator 22. The actuator 22 is configured to be moved in a desired direction without a need to disengage the actuator 22 prior to movement. The actuator 22 is free to slide proximally or distally relative to the handle 10 and a location of the actuator 22 remains fixed by interaction of the elongated medical device with a friction brake 44 as discussed below.

In some embodiments, the elongated medical device includes an outer member 24 connected to a distal end 26 of the handle 10, and an inner member 28 (FIGS. 3 and 4) longitudinally moveable within the outer member 24. Movement of the inner member 28 relative to the outer member 24 causes the elongated medical device to change shape or arrangement. In some embodiments, the elongated medical device moves from a straight to a curved configuration. In some embodiments, the elongated medical device moves from a stored to an expanded configuration.

The actuator 22 is configured to be moved by a user's thumb and includes a receiver 30 that extends into the inner cavity 16. The receiver 30 is connected to the inner member 28. The inner member 28 can connected to the receiver 30 using adhesives, mechanical couplers, or other means for joining the inner member 28 to the receiver 30. As the actuator 22 is moved longitudinally with respect to the housing 10, the receiver 30, engaged with the inner member 28, moves the inner member 28 longitudinally within the outer member 24.

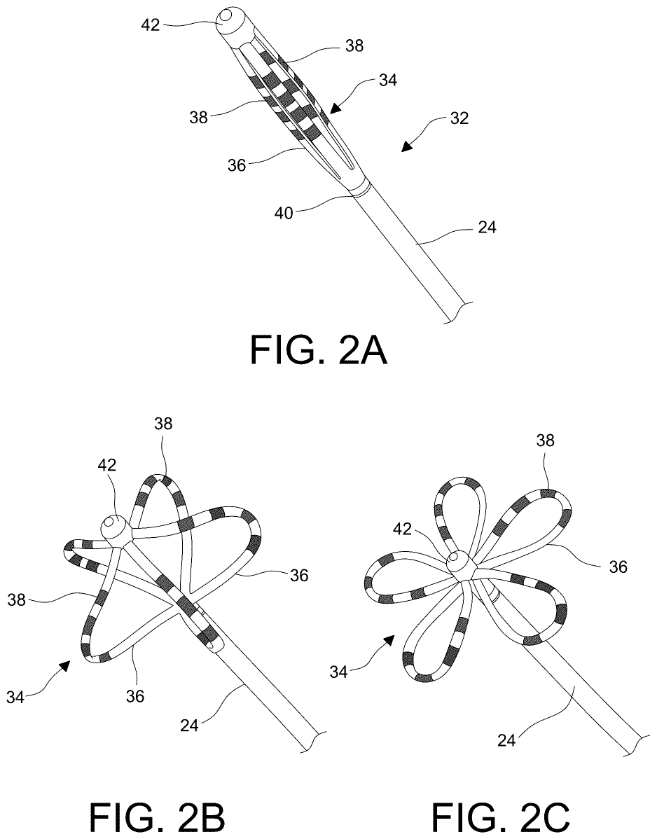

FIG. 2A illustrates an elongated medical device in the form of an ablation catheter 32. The ablation catheter 32 includes a distal portion 34. The distal portion 34 includes a plurality of splines 36 each having at least one electrode 38. The plurality of splines 36 form a basket structure that positions the at least one electrode 38 against a tissue surface within a patent. The embodiment illustrated in FIG. 2A-2C includes five splines 36, and each of the five splines 36 includes four electrodes 38. Other embodiments include more or less splines 36 and more or less electrodes 38 to facilitate use in various locations of a patient and to achieve a desired purpose. The at least one electrode 38 can take the form of ablation electrodes, mapping electrodes, pacing electrodes, sensing electrodes, or cauterizing electrodes depending on a procedure to be performed.

The outer member 24 of the ablation catheter 32 includes a distal end 40 that is attached to a proximal end of the plurality of splines 36. A distal end of the plurality of splines 36 is attached to a cap 42. In some embodiments, the cap 42 is a separate component. In other embodiments, the cap 42 is formed by molding together the plurality of splines 36. The cap 42 is attached to a distal end of the inner member 28 such that retraction of the inner member causes the plurality of splines 36 to move from a stored configuration as illustrated in FIG. 2A to an expanded configuration as illustrated in FIGS. 2B and 2C.

In FIG. 2A, the stored configuration of the distal portion 34 corresponds to the actuator 22 being in a most distal position on the handle 10. In this arrangement, the inner member 28 is also in a most distal position such that the plurality of splines 36 are substantially parallel to a longitudinal axis of the ablation catheter 32.

In FIG. 2B, the distal portion 34 of the ablation catheter 32 is in an expanded configuration where the splines 36 form a bow shape. This arrangement corresponds to the actuator 22 being retracted proximally from the most distal position, which in turn retracts the inner member 28.

In FIG. 2C, the distal portion 34 of the ablation catheter 32 is in an expanded configuration where the splines 36 form a petal configuration. This arrangement corresponds to the actuator 22 being retracted to a most proximal position on the handle 10, and the inner member 28 it its most proximal position.

The actuator 22 is capable of being moved from a most proximal position to a most distal position, and vice versa, by a user pushing or pulling the actuator. The distal portion 34 of the ablation catheter 32 moves between the stored configuration to the petal configuration as the actuator 22 is translated. A user is able to leave the actuator 22 in any location between the most distal positron and the most proximal position in order to expand the splines 36 a desired amount between the stored configuration and the petal configuration. The actuator 22 remains in any desired location by interaction of the inner member 28 with a friction brake 44. The friction brake 44 includes a polymeric body that is configured to apply constant friction force to the inner member 28 such that a position of the actuator 22 is maintained when a user releases the actuator 22. The polymeric body is formed of a low friction material, for example acetal. The low friction material is configured to minimize any stick-slip phenomenon between the polymeric body and the inner member 28, such that the inner member 28 moves consistently through the friction brake 44 as the actuator 22 is moved but remains stationary when the actuator 22 is released.

As illustrated in FIG. 4, the friction brake 44 is located within the inner cavity 16 and is aligned with the inner member 28. The friction brake 44 is supported within the inner cavity 16 by at least one column 46. As shown in FIG. 4, the at least one column 46 includes a plurality of columns 46 that retain the friction brake 44. Two columns 46 are positioned proximally of the friction brake 44 and two are positioned distally of the friction brake 44. The at least one column 46 extends from an inner surface of the right housing portion 20 to secure the friction brake 44.

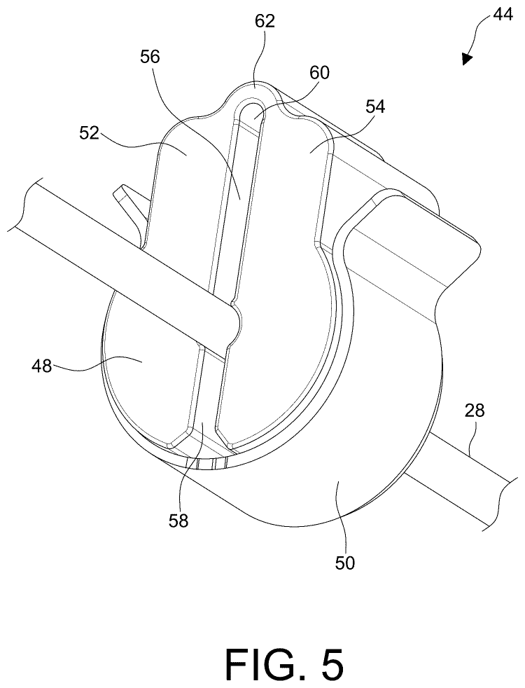

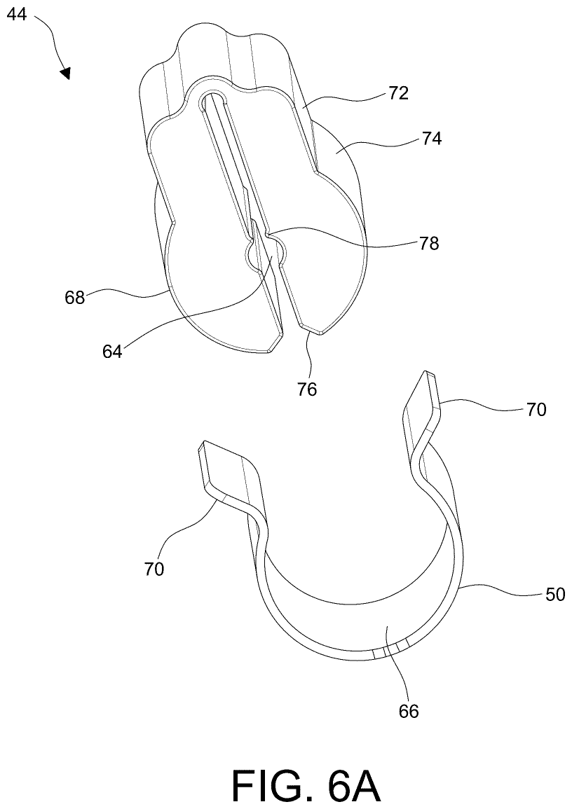

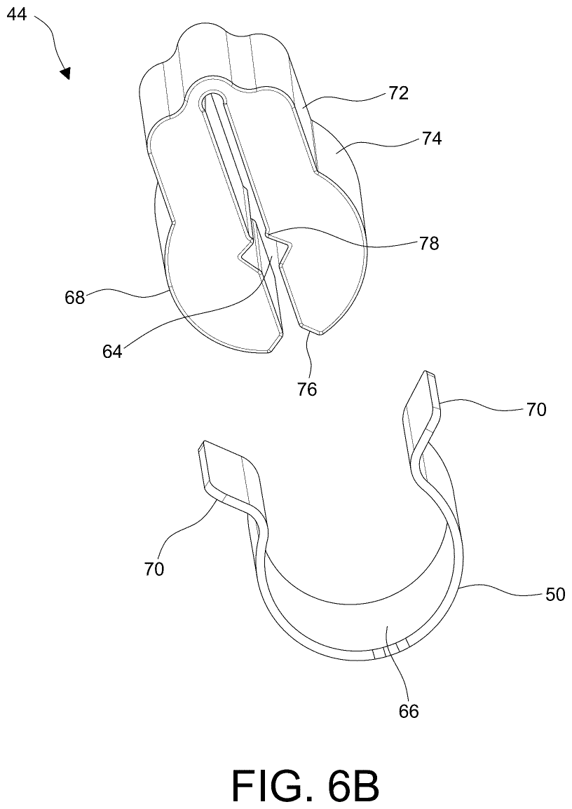

FIG. 5 is a perspective view of first embodiment of a friction brake 44 for use with the handle 10 of FIG. 1. FIG. 6 are exploded perspective views of the friction brake 44 of FIG. 5. The friction brake 44 includes a polymeric body 48 and a clamp 50 that holds the polymeric body 48. The polymeric body 48 has a first arm 52 and a second arm 54 separated by a slot 56. The slot 56 includes a first open end 58 and a second closed end 60. A flare is 76 is located adjacent the first open end 58 of the slot 56 for aiding in the introduction of the inner member into the polymeric body 48. The first arm 52 and the second arm 54 are connected by a hinge 62 adjacent the second end 60 of the slot 56. As illustrated, the hinge 62 protrudes above a surface of the polymeric body 48. A channel 64 configured to receive the elongated medical device, specifically the inner member 28, traverses the slot 56. The channel 64 has a circular cross-section configured to surround the inner member 28. A protrusion 78 is positioned adjacent the channel 64 and extends into the slot 56. The protrusion 78 acts as a stop when placing the polymeric body 48 onto the inner member 28. As shown in FIG. 6A, the protrusion 78 is curved with a radius of curvature similar to that of the inner member 28. In various embodiments, the radius of curvature is between equal to and up to 15% less than that of the inner member 28. As shown in FIG. 6B, the channel 64 is angular (e.g., diamond-shaped) rather than curved. In certain embodiments, the channel 64 may have an angle of between about 60 and about 120 degrees.

The clamp 50 is configured to support the polymeric body 48 and aids in keeping the first arm 52 and the second arm 54 from moving apart. The clamp includes a portion 66 having a circular cross-section. This portion 66 surrounds a corresponding cylindrical portion 68 of the polymeric body. The first arm 52 and the second arm 54 each include an outer surface having a straight portion 72 and a curved portion 74. The curved portions 74 form an outer surface of the cylindrical portion 68. The clamp 50 also includes at least one wing 70 that extends away from the polymeric body 48. The at least one wing 70 extends away from the polymeric body 48 at an intersection of the curved portion 74 and the straight portion 72. In some embodiments, the clamp 50 is formed of spring steel.

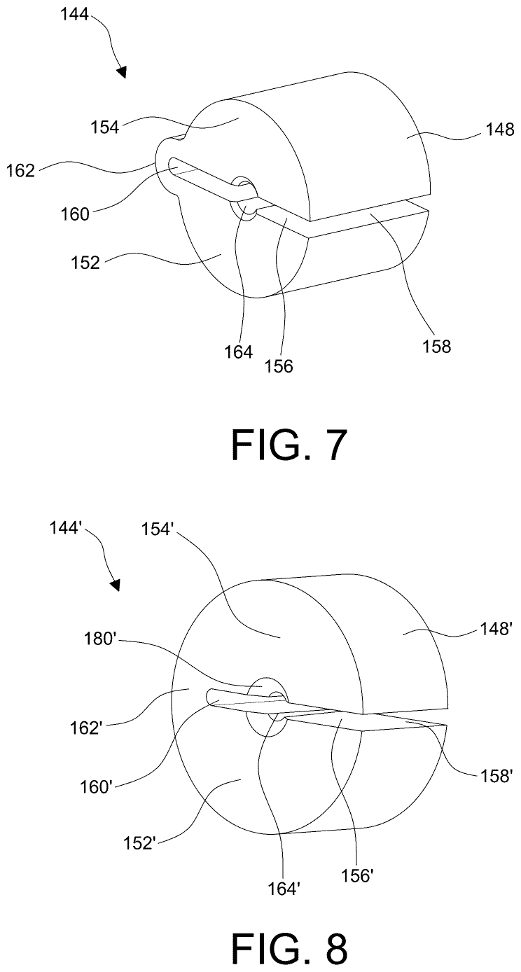

FIG. 7 is a perspective view of second embodiment of a friction brake 144 for use with the handle 10 of FIG. 1. The friction brake 144 includes a polymeric body 148 having a first arm 152 and a second arm 154 separated by a slot 156. The slot 156 includes a first open end 158 and a second closed end 160. The first open end 158 is formed in a curved surface of the polymeric body 148. The first arm 152 and the second arm 154 are connected by a hinge 162 adjacent the second end 160 of the slot 156. The hinge 162 protrudes above a surface of the polymeric body 148. A channel 164 configured to receive the elongated medical device, specifically the inner member 28, traverses the slot 156. The channel 164 has a circular cross-section configured to surround the inner member 28. The polymeric body 148 has a cylindrical shape aside from the hinge 162. A clamp (not shown) may surround a portion of the polymeric body 148.

FIG. 8 is a perspective view of third embodiment of a friction brake 144′ for use with the handle 10 of FIG. 1. The friction brake 144′ includes a polymeric body 148′ having a first arm 152′ and a second arm 154′ separated by a slot 156′. The slot 156′ includes a first open end 158′ and a second closed end 160′. The first open end 158′ is formed in curved surface of the polymeric body 148′. The first arm 152′ and the second arm 154′ are connected by a hinge 162′ adjacent the second end 160′ of the slot 156′. The hinge 162′ is formed by a portion of the polymeric body 148′ and does not extend outside of the polymeric body 148′. A channel 164′ configured to receive the elongated medical device, specifically the inner member 28, traverses the slot 156′. The channel 164′ includes a chamfer 180′ and has a circular cross-section configured to surround the inner member 28. A clamp (not shown) may surround a portion of the polymeric body 148′.

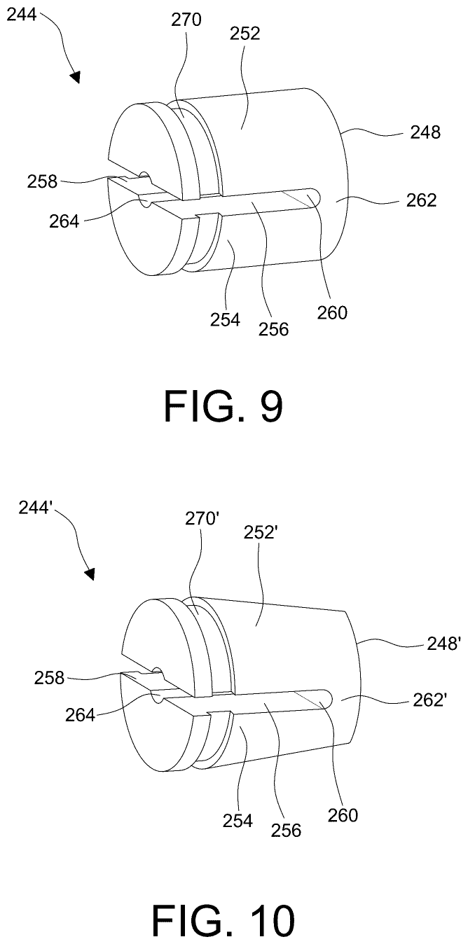

FIG. 9 is a perspective view of fourth embodiment of a friction brake 244 for use with the handle 10 of FIG. 1. The friction brake 244 includes a polymeric body 248 having a first arm 252 and a second arm 254 separated by a slot 256. The slot 256 includes a first open end 258 and a second closed end 260. The first open end 258 is formed in a planar surface of the polymeric body 248. The first arm 252 and the second arm 254 are connected by a hinge 262 adjacent the second end 260 of the slot 256. The hinge 262 is formed by a portion of the polymeric body 248 and does not extend outside of the polymeric body 248. A channel 264 configured to receive the elongated medical device, specifically the inner member 28, traverses the slot 256. The channel 264 begins at the first open end 258 of the slot 256 and extends through the hinge 262. The channel 264 has a circular cross-section configured to surround the inner member 28. The polymeric body 248 includes a groove 270 configured to receive a clamp (not shown) to aid in keeping the first arm 252 and the second arm 254 stationary relative to one another.

FIG. 10 is a perspective view of fifth embodiment of a friction brake 244′ for use with the handle 10 of FIG. 1. The friction brake 244′ includes a polymeric body 248′ having a first arm 252′ and a second arm 254′ separated by a slot 256′. The slot 256′ includes a first open end 258′ and a second closed end 260′. The first open end 258′ is formed in a planar surface of the polymeric body 248′. The first arm 252′ and the second arm 254′ are connected by a hinge 262′ adjacent the second end 260′ of the slot 256′. The hinge 262′ is formed by a portion of the polymeric body 248′ and does not extend outside of the polymeric body 248′. A channel 264′ configured to receive the elongated medical device, specifically the inner member 28, traverses the slot 256′. The channel 264′ begins at the first open end 258′ of the slot 256′ and extends through the hinge 262′. The channel 264′ has a circular cross-section configured to surround the inner member 28. The polymeric body 248′ includes a groove 270′ configured to receive a clamp (not shown) to aid in keeping the first arm 252′ and the second arm 254′ stationary relative to one another. Additionally, the polymeric body 248′ includes an outer dimeter that tapers along a longitudinal axis of the channel 264′.

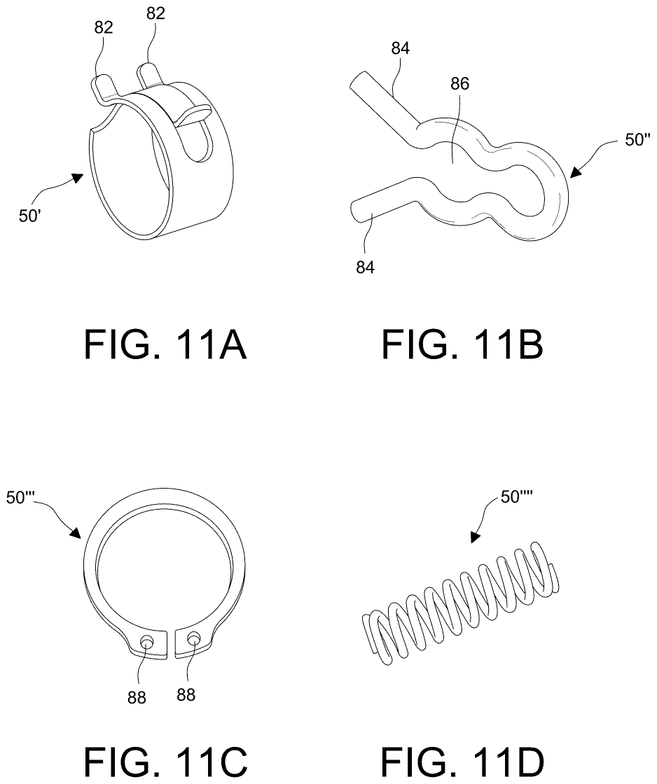

FIG. 11A-11D are perspective views of various clamps 50′, 50″, 50′″, 50″″ for use with a friction brake, in accordance with embodiments of the disclosure. The claims 50′ illustrated in FIG. 11A-11D may be configured to surround all or a portion of a polymeric body. As illustrated in FIG. 11A, the clamp 50′ includes tabs 82 to aid in opening and positioning of the clamp 50′ onto a polymeric body. FIG. 11B illustrates a clamp 50′ having arms 84 including a central region 86 that contacts a polymeric body. FIG. 11C illustrates a clamp 50′″ having eyes 88 which can be used to aid in opening and positioning of the claim 50′″ onto a polymeric body. FIG. 11D illustrates a claim 50″″ configured as a coiled spring which surrounds at least a portion of a polymeric body.

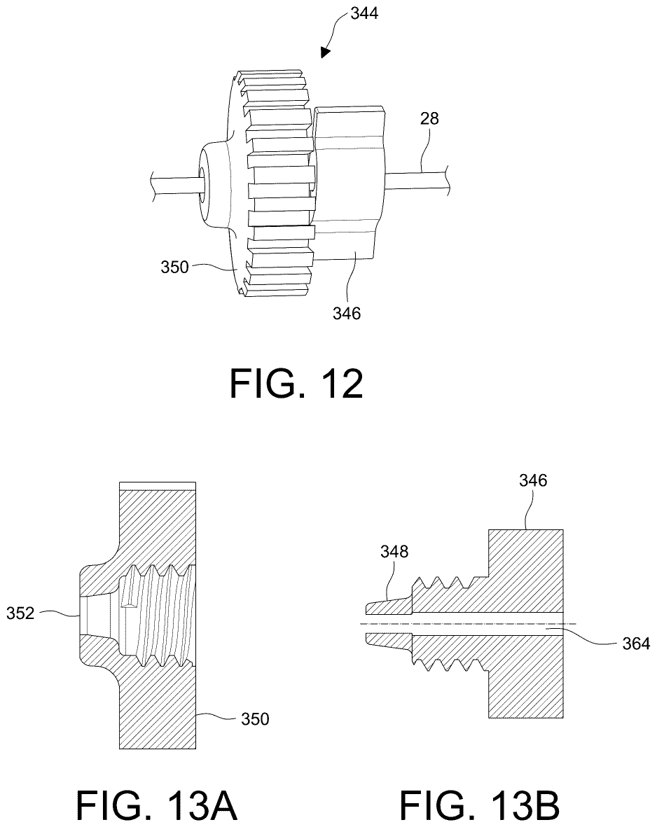

FIGS. 12, 13A, and 13B illustrate an embodiment of a friction brake 344 having an adjustable braking level for use with the handle 80 of FIG. 1. The friction brake 344 of FIGS. 12, 13A, and 13B utilize a collet 346 in combination with a cap 350. The collet 344 includes fingers 348 that surround one end of a channel 364. The channel 364 is configured to receive the inner member 28. The cap 350 is threadedly engaged to the collet 346 and includes an angled inner surface 352. As the cap 350 is twisted onto the collet 346, the angled inner surface 352 of the cap 350 presses against the fingers 348 and causes the fingers 348 to deflect into the channel 364. The greater the deflection of the fingers 348 into the channel, the greater the friction force applied to the inner member 28. This arrangement allows a user to adjust the level of friction applied by the friction brake 344 to the inner member 28. If the actuator 22 seems to move after the user places the actuator 22 in a desired location, then the user can increase the friction applied by the friction brake 344. If the actuator 22 fail to move because the brake 344 is applied too much friction to the inner member 28, then the user can reduce the level of friction by unscrewing the cap 350 until a desired level of friction is achieved.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A friction brake for use in a medical device, the friction brake comprising:
a polymeric body including a first arm and a second arm separated by a slot, the slot having a first open end and a second closed end, the first arm and the second arm being connected by a hinge adjacent the second end of the slot, a channel traversing the slot, the channel configured to receive an elongated medical device;
wherein the polymeric body is configured to apply constant friction force to the elongated medical device and is formed of a low friction material configured to minimize any stick-slip phenomenon between the polymeric body and the elongated medical device.

2. The friction brake of claim 1, further comprising a clamp configured to hold the polymeric body.

3. The friction brake of claim 1, wherein the channel comprises a diamond-shaped cross-section.

4. The friction brake of claim 1, wherein the clamp includes a portion having a circular cross-section.

5. The friction brake of claim 1, wherein the polymeric body includes a cylindrical portion.

6. The friction brake of claim 1, wherein the material comprises acetal.

7. The friction brake of claim 1, wherein the hinge protrudes above a surface of the polymeric body.

8. The friction brake of claim 1, further comprising a flare adjacent the first open end of the slot.

9. The friction brake of claim 1, wherein the first arm and the second arm include an outer surface having a straight portion and a curved portion.

10. The friction brake of claim 1, wherein the polymeric body further comprises a protrusion positioned adjacent the channel and extending into the slot.

11. The friction brake of claim 2, wherein the clamp includes at least one wing that extends away from the polymeric body.

12. The friction brake of claim 2, wherein the polymeric body includes a groove configured to receive the clamp.

13. The friction brake of claim 1, wherein the polymeric body includes an outer dimeter that tapers along a longitudinal axis of the channel.

14. The friction brake of claim 1, wherein the channel passes through the hinge.

15. An ablation device, the ablation device comprising:
a handle including an actuator;
an outer member including a proximal portion connected to the handle and a distal portion having at least one electrode;
an inner member movable within the outer member using the actuator, a shape of the distal portion being changeable by moving the inner member; and
a friction brake including a polymeric body, the polymeric body including a first arm and a second arm separated by a slot, the slot having a first open end and a second closed end, the first arm and the second arm being connected by a hinge adjacent the second end of the slot, and a channel traversing the slot, the inner member received in and movable through the channel;
wherein the polymeric body is configured to apply constant friction force to the inner member such that a position of the actuator is maintained.

16. The ablation device of claim 15, wherein the friction brake includes a clamp configured to hold the polymeric body.

17. The ablation device of claim 15, wherein the distal portion is changeable from a stored configuration to an expanded configuration.

18. The ablation device of claim 15, wherein the distal portion is changeable from a straight configuration to a curved configuration.

19. A handle for use in a medical procedure, the handle comprising:
a housing having an outer surface and an inner cavity;
an actuator moveable relative to the housing, the actuator configured to translate an elongated medical device;
a friction brake located in the inner cavity, the friction brake including a polymeric body, the polymeric body including a first arm and a second arm separated by a slot, the slot having a first open end and a second closed end, the first arm and the second arm being connected by a hinge adjacent the second end of the slot, and a channel traversing the slot that receives the elongated medical device;
wherein the polymeric body is configured to apply constant friction force to the elongated medical device such that a position of the actuator is maintained.

20. The handle of claim 19, comprising at least one column configured to support the friction brake within the inner cavity.
